(19) United States
(12) Patent Application Publication
(10) Pub. No.: US 2024/0108290 A1
(43) Pub. Date: Apr. 4, 2024

(54) WEARABLE DEVICE FOR MEASURING MULTIPLE BIOSIGNALS, AND ARTIFICIAL-INTELLIGENCE-BASED REMOTE MONITORING SYSTEM USING SAME

(71) Applicant: Medifarmsoft Co., Ltd., Seoul (KR)

(72) Inventor: Jae Hu JEON, Seoul (KR)

(73) Assignee: Medifarmsoft Co., Ltd., Seoul (KR)

(21) Appl. No.: 18/276,491

(22) PCT Filed: Dec. 14, 2021
(86) PCT No.: PCT/KR2021/018949

§ 371 Date: Aug. 9, 2023

(30) Foreign Application Priority Data
Feb. 9, 2021 (KR) 10-2021-0018514
Feb. 9, 2021 (KR) 10-2021-0063275

(51) Int. Cl. A61B 5/00; A61B 5/0205; A61B 5/318

(52) U.S. Cl. CPC A61B 5/7275; A61B 5/002; A61B 5/02055; A61B 5/318; A61B 5/681; A61B 5/746; A61B 5/0816

(57) ABSTRACT

The present invention relates to a wearable device for measuring multiple biosignals, and a remote monitoring system using same, and the wearable device for measuring multiple biosignals relates to a biometric data measurement system, and comprises: a main body having one or more external ports; a wrist band connected to the main body and wound around the wrist of a user; and one or more external sensors connected to the external ports. The wearable device for measuring multiple biosignals comprises: one or more internal sensors for measuring multiple biosignals; a wireless communication unit for transmitting, to the outside, the multiple biosignals obtained from the internal sensors or the external sensors; a user interface unit for displaying a notification message indicating the multi-biosignal measurement and receiving an input of user data; a sensor control unit, which amplifies output signals of the internal sensors, converts the amplified signals into digital signals, and detects whether the external sensor is connected to the external port to inactivate an internal sensor for measuring the same biosignal that the external port measures when the external sensor is connected to the external port; and a central control unit for supplying multi-biosignal data from the sensor control unit to the wireless communication unit, and transmitting a notification message received from the wireless communication unit to a display of the user interface unit.

### TECHNICAL FIELD

The present invention relates to a multi-biosignal measuring device, which is directly wearable by a user, and an artificial intelligence (AI)-based remote monitoring device using the same.

### BACKGROUND ART

Recently, technologies of various biosensors for measuring biosignals have become remarkably sophisticated and miniaturized. In addition, there is the development of communication technology capable of transmitting data at high speed, and indoor and outdoor positioning technology, and the emergence of medical devices capable of utilizing accumulated previous hospital medical records or biometric information for analysis and diagnosis through artificial intelligence (AI) using big data.

However, since patient monitoring devices currently used in hospitals are fixed types and have complicated functions, they are available for use and analysis only by experts with relevant knowledge.

Therefore, the present invention relates to a wearable multi-biometric measuring device capable of measuring biometric information using small sensor technology, communication technology, and AI technology at a remote site in an aware state and an unaware state, and monitoring abnormalities through measurement data and coping with the abnormalities, and an AI-based remote monitoring system using the same.

As a related art, there are disclosed Korean Registered Patent No. 10-2210242 (Jan. 26, 2021), Korean Registered Patent No. 10-2210215 (Jan. 26, 2021), Korean Registered Patent No. 10-2197112 (Dec. 31, 2020), Korean Registered Patent No. 10-2208759 (Jan. 22, 2021), and the like.

### DISCLOSURE

### Technical Problem

Through measurement of current biometric data of a patient, the current severity of the patient's symptoms or the prognosis trend after treatment may be determined. In general, a nurse in a medical institution periodically measures a blood pressure, a pulse rate, a body temperature, an electrocardiogram (ECG), an oxygen saturation, etc. of a patient for measurement of biometric data, and manually writes the measured value. In order to measure the patient's biometric data, nursing personnel and professional examination personnel are required, and it takes a long time to measure and analyze biometric data, and particularly, in the case of discharged patients who need prognosis management, prognosis management is possible only through a revisit to the hospital.

In addition, for some biometric data, e.g., an ECG for determining the presence or absence of a heart disease, there is a shortage of specialists for reading ECG data except for large hospitals, which results in a long time spent on reading and analyzing the data.

At various sites, such as a cardiology department waiting room and an emergency room of a hospital, a screening clinic, a life treatment center/quarantine facility, an infectious disease control site, a medical blind spot, and the like, vital signs (blood pressure, pulse rate, respiratory rate, and temperature) test are essential. In the case of patients with severe or moderately severe symptoms, oxygen saturation tests and ECGs are additionally required.

Emerging infectious diseases, for example, Middle East respiratory syndrome (MERS) or coronavirus disease (COVID-19) are spreading worldwide. The spread of infectious diseases has led to extreme fatigue of medical workers, and a severe shortage of medical devices. In this situation, repetitive tests of biometric data pose a high risk of fatigue accumulation and infection exposure accidents for medical staff.

Patient monitors currently widely used in hospitals are fixed types, which are large, heavyweight, not easy to carry, and expensive, so there are a number of limitations in their use. In the case of hospitalized patients, it is difficult to measure a patient when the patient leaves the hospital room even for a while, and especially in the case of discharged patients who need prognosis monitoring, measurement and diagnosis are performed only through a revisit to the hospital, which results in significant inconvenience.

Therefore, the present invention relates to a wearable multi-biosignal measuring device provided to resolve the issues of the related art, and having a function of measuring multiple biosignals in a wearable form in an aware state and an unaware state for not only a hospitalized patient but also a discharged patient who needs prognosis management, remotely monitoring the measurement in real time, and in response to an abnormal situation being detected, allowing an alarm to be issued such that an immediate response is taken, and a remote monitoring system using the same.

The wearable multi-biosignal measuring device according to the present invention is individually worn by a hospitalized patient and a discharged patient such that multiple biosignals are measured in an aware state and an unaware state and transmitted for remote monitoring, and thus the trend of the patient's biosignal change may be monitored in real time, and depending on symptoms or severity, selectively expanded to an external high-precision sensor and worn, such that high-quality biosignals of hospitalized patients or discharged patients are acquired and monitored.

The wearable multi-biosignal measuring device according to the present invention allows a remote monitoring system to automatically or manually control a sensor or a measurement cycle of the wearable multi-biometric measuring device, and thus customized biometric information according to the patient's condition may be measured.

The remote monitoring system according to the present invention is implemented to, upon deviation from a normal reference or a danger being detected through a prognosis prediction analysis server, which uses a heart disease analysis result through an AI ECG analysis server and a medical history of a hospital information system as well as, multiple pieces of biometric information, issue an alarm, and transmit an alarm signal to medical staff or patients.

The technical objectives of the present invention are not limited to the above, and other objectives that are not described above may become apparent to those of ordinary skill in the art based on the following descriptions.

### Technical Solution

A wearable device for measuring multiple biosignals according to an embodiment of the present invention includes: a main body having one or more external ports; a wristband connected to the main body and wound around a wrist of a user; and one or more external sensors connected to the external ports.

The main body includes: one or more built-in sensors configured to measure biosignals; a wireless communication unit configured to transmit multiple biosignals obtained from the built-in sensor or the external sensor to the outside; a user interface unit configured to display a notification message instructing measurement of the multiple biosignals and receive user data; a sensor control unit configured to amplify an output signal of the built-in sensor and convert the amplified signal into a digital signal, detect whether the external sensor is connected to the external port, and when the external sensor is connected to the external port, disable the built-in sensor that measures the same biosignal as the external port; and a central control unit configured to supply multi-biosignal data from the sensor control unit to the wireless communication unit and transmit a notification message received from the wireless communication unit to a display of the user interface unit.

A remote monitoring system according to an embodiment of the present invention is connected to the wearable multi-biosignal measuring device through a wireless communication network. When the remote monitoring system transmits a biosignal measurement profile message to the wearable multi-biosignal measuring device, the wearable multi-biosignal measuring device transmits multi-biometric information associated with a type and a measurement cycle of a sensor, quarantine area departure information according to content of the biosignal measurement profile message, and the like to the remote monitoring system through a gateway.

The remote monitoring system enables real-time monitoring of received multiple biosignals, requests reading by deep learning artificial intelligence through a heart disease analysis server and a prognosis prediction analysis server to proceed with heart disease or prognosis analysis, and in response to deviation from a preset range of alarm reference values, transmits an alarm message to a monitoring system of a hospital and mobile terminals of medical staff and a patient.

### Advantageous Effects

The present invention can measure multiple biosignals in an unaware state using a wearable multi-biosignal measuring device individually provided to a severely ill hospitalized patient or a discharged patient requiring prognosis management, and allow the patient to measure required biosignals by himself/herself. In addition, the remote monitoring system is connected to the wearable multi-biosignal measuring device through a wireless communication network to continuously monitoring multiple biosignals received from the wearable multi-biosignal measuring device in real time, and thus in the case of a severely ill hospitalized patient, measurement and monitoring of multiple biosignals can be performed in a non-contact manner, and in the case of a discharged patient who requires prognosis management, prognosis management can be remotely performed without visiting the hospital.

The present invention can significantly reduce the fatigue of medical staff who need to periodically repeat tests of four vital signs (blood pressure, pulse rate, respiratory rate, and body temperature) of general patients, and tests of an oxygen saturation and an electrocardiogram (ECG) for patients with severe or moderately-severe symptoms, and also can minimize contact between patients and medical staff, thereby minimizing the risk of infection of Middle East respiratory syndrome (MERS), coronavirus disease (COVID-19), or the like.

The present invention can improve nursing work through automation of multi-biosignal measurement and recording tasks for hospitalized patients and discharged patients.

The present invention can enable remote monitoring of discharged patients, thereby saving time and money due to reducing the number of hospital visits, and also preventing secondary infection of infectious diseases.

The remote monitoring system according to the present invention can allow medical staff to use an external high-precision sensor or automatically or manually change and set a measurement sensor or a measurement cycle when continuously remotely monitoring the status and changes of basic biosignals of hospitalized patients and discharged patients, thereby achieving close patient-customized monitoring.

The remote monitoring system according to the present invention can issue an alarm in a monitoring system terminal upon exceeding an alarm reference value for each patient/biometric data, and transmit alarm information to the medical staff in charge and the on-call doctor, thereby performing an immediate response on a discharged patient, who needs emergency treatment due to a worsening condition.

The remote monitoring system according to the present invention can provide services, such as assessing a sleep state, checking an exercise amount, notifying a fall, and analyzing snoring using the wearable multi-biosignal measuring device, and various additional services, such as, checking whether a treatment effort is being carried out properly by a patient, who is prescribed exercise, by monitoring exercise amount information, checking whether self-quarantine is being complied with in real time through location information of a patient, and the like.

The effects of the present invention are not limited to those described above, and other effects that are not described above will be clearly understood by those skilled in the art from the above detailed description.

### DESCRIPTION OF DRAWINGS

FIG. 1 is a service conceptual diagram of a wearable multi-biosignal measuring device and a remote monitoring system according to an embodiment of the present invention.

FIG. 2 is a block diagram showing a hardware configuration of a wearable multi-biosignal measuring device according to an embodiment of the present invention.

FIG. 3 is a diagram showing a wearable multi-biosignal measuring device in the form of a band according to an embodiment of the present invention.

FIG. 4 is a flowchart showing a power on sequence immediately after power is applied to the wearable multi-biosignal measuring device illustrated in FIG. 3.

FIGS. 5A and 5B are flowcharts showing interrupt service routines depending on whether external sensors are connected.

FIG. 6 is a diagram showing a configuration of a remote monitoring system in detail.

FIG. 7 is a diagram showing an artificial intelligence-based diagnosis algorithm for internal or external electrocardiogram (ECG) data.

FIG. 8 is a diagram showing a configuration of a prognosis prediction server that informs a prognosis prediction result for a disease by learning multiple biosignals, a heart disease analysis result, and a medical history.

### BEST MODES OF THE INVENTION

Hereinafter, advantages, features, and ways to achieve them will become readily apparent with reference to the following detailed description of embodiments when considered in conjunction with the accompanying drawings. The present invention is not limited to the embodiments disclosed below, and may be embodied in various forms. The embodiments to be described below are only embodiments provided to complete the disclosure of the present invention and help those skilled in the art to completely understand the scope of the present invention, and the present invention is defined only by the scope of the appended claims.

Since the shapes, sizes, ratios, angles, numbers, and the like disclosed in the drawings for describing the embodiments of the present invention are illustrative, the present invention is not limited to the details shown in the drawings. Throughout the specification, like reference numerals refer to substantially like elements. In addition, in the description of the present invention, when it is determined that a detailed description of known related art unnecessarily obscures the subject matter of the present invention, the detailed description will be omitted.

When “is provided with,” “includes,” “has,” “consists of,” and the like mentioned in this specification are used, other parts may be added unless “— only” is used. When an element is expressed in the singular, it may be interpreted in the plural unless otherwise specified.

In interpreting the constituent elements, it is construed that they include an error range without any explicit description.

In the case of a description of a positional relationship, for example, when the positional relationship between two parts is described as “on,” “on top,” “under,” or “next to,” elements that are not used together with terms “immediately” or “directly” may have one or more other intervening elements therebetween.

Although first, second, and the like may be used to distinguish elements, the function or structure of the elements is not limited to the ordinal number or the name attached to the front of the element.

The following embodiments may be partially or entirely combined with each other, and technically enable various interworking and driving operations. Each embodiment may be implemented independently of each other or may be implemented together in an associative relationship.

The wearable multi-biosignal measuring device according to the present invention enables measurement of various biosignals, such as a pulse wave, a body temperature, a blood pressure, and an exercise amount as well as an electrocardiogram (ECG), and enables more sophisticated biosignal collection according to a symptom or severity of illness of a patient selectively through an expandable ECG electrode or a high-precision external sensor.

In addition, the remote monitoring system is a system that automatically sets a sensor or a measurement cycle of a wearable multi-biometric information measuring device through an automatic measurement setting function such that biosignals are received in an aware state or an unaware state through a gateway and remotely monitored, or provides a real-time alarm through a heart disease analysis server or a prognosis prediction analysis server.

Therefore, through the wearable multi-biosignal measuring device and the remote monitoring system according to the present invention, a severely ill hospitalized patient or a discharged patient who requires prognostic monitoring may be subject to real-time monitoring of biosignals, and upon generation of an abnormal signal, an immediate response may be taken, and additionally, a fall, the amount of sleep, sleep apnea, and the amount of exercise of a patient may be checked, and the departure of self-quarantined people from a quarantine area may be monitored.

Hereinafter, various embodiments of the present invention will be described in detail with reference to the accompanying drawings.

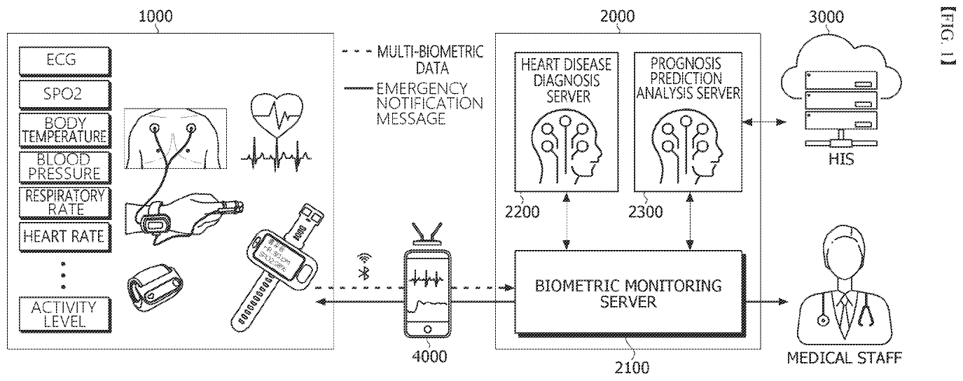

FIG. 1 is a service conceptual diagram of a wearable multi-biosignal measuring device 1000 and a remote monitoring system 2000 according to an embodiment of the present invention.

Referring to FIG. 1, the present invention may provide a wearable multi-biosignal measuring device 1000 to a severely ill hospitalized patient or a discharged patient who requires prognosis management. The wearable multi-biosignal measuring device 1000 measures biosignals, such as a blood pressure, a heart rate, a respiratory rate, a body temperature, an ECG, an oxygen saturation, and an exercise amount, or location information in an aware state and an unaware state, and transmits the biosignals or the location information to the remote monitoring system 2000 through a gateway 4000.

The wearable multi-biosignal measuring device 1000 operates based on a sensor, a measurement cycle, and a quarantine range as set according to the content of a biosignal measurement profile message set and transmitted by the remote monitoring system 2000, and measures biosignals and whether a departure from a quarantine area has occurred and transmits the result.

In the remote monitoring system 2000, the wearable multi-biosignal measuring device 1000 is device-registered to a severely ill hospitalized patient or a discharged patient, who requires prognosis monitoring, through a near field communication (NFC)/radio frequency identification (RFID) reader, and in this case, a biosignal measurement profile message according to the disease is initialized and transmitted, and information about the patient is recorded in the wearable multi-biosignal measuring device 1000.

Thereafter, the remote monitoring system 2000, when there is a need to change details associated with measurement of various biosignals according to a prognosis or a request of a medical staff, updates a multi-signal measurement profile message and transmits the updated multi-signal measurement profile message to the wearable multi-biosignal measuring device 1000.

The multi-signal measurement profile message includes information about the type of biosignal that needs to be measured, each measurement cycle, and the range of a quarantine area for each patient, and is set to an initial value when the device is registered, but may be changed at a later time according to the prognosis or the determination of the medical staff.

The remote monitoring system 2000 may include a heart disease diagnosis server 2200, a prognosis prediction analysis server 2300, and a biometric monitoring server 2100.

The heart disease diagnosis server 2200 diagnoses various heart diseases using information obtained through the waveform analysis of ECG signals received from the wearable multi-biosignal measuring device 1000 and learning data obtained through image learning by deep learning, and transmits the result to the biometric monitoring server 2100.

The prognosis prediction analysis server 2300 transmits, to the biometric monitoring server 2100, a prognosis prediction analysis result corresponding to current biometric information based on the content learned from a medical history from a hospital information system (HIS) 3000, a disease, a treatment and biometric information of a patient by deep learning.

The biometric monitoring server 2100 of the remote monitoring system 2000 performs a function of displaying biometric information to enable real-time monitoring of biometric information from the wearable multi-biosignal measuring device 1000, and transmits such biosignals to the heart disease analysis server 2200 and the prognosis prediction analysis server 2300.

In addition, the biometric monitoring server 2100 of the remote monitoring system 2000 receives a heart disease analysis result and a prognosis prediction result from the heart disease analysis server 2200 and the prognosis prediction analysis server 2300, respectively, and issues various alarms in response to a deviation from a normal reference value.

The alarms may be directly presented on the biometric monitoring server 2100 of the remote monitoring system 2000 or transmitted to the medical staff or doctor on duty and the wearable multi-biosignal measuring device 1000 worn by the patient to rapidly inform the patient's dangerous situation.

In addition, the remote monitoring system according to the present invention allows a biometric information measurement history of a patient to be checked in an application for medical staff or an application for patients, provides various types of diagnosis information, such as an activity level, a sleep time, whether a sleep apnea symptom is present, heart disease prediction information through an AI-based ECG reading result, and the like, and also provides a messaging function that allows a message entered by medical staff or a response message of a patient to be received and transmitted.

In the present invention, the wearable multi-biosignal measuring device 1000 worn by each patient and the remote monitoring system 2000 may be used such that repetitive measurement and recording of biometric information for each patient are automated and real-time monitoring and alarm services are provided, thereby enhancing the efficiency of hospital operations and reducing unnecessary hospital visits of a patient who requires prognosis management after discharge, and dramatically improving nursing work, and preventing secondary infections and spread in the event of an infectious disease.

The remote monitoring system 2000 may analyze data received from the wearable multi-biosignal measuring device 1000 in real time to monitor an activity level, sleep, sleep apnea or a fall of each patient, or may use location information to identify the location of the patient and provide monitoring services about a departure from a quarantined area.

When the wearable multi-biosignal measuring device 1000 acquires data from a built-in or external sensor and transmits the data to the remote monitoring system 2000 through the gateway 4000, the biometric monitoring server 2100 of the remote monitoring system 2000 may monitor a biosignal trend in real time, and upon determining that there is an abnormality through analysis results of the heart disease analysis server 2001 and the prognosis prediction analysis server 2300, issue an alarm, and the result may be immediately delivered to the medical staff as well as the patient so that it is informed and appropriate measures should be taken.

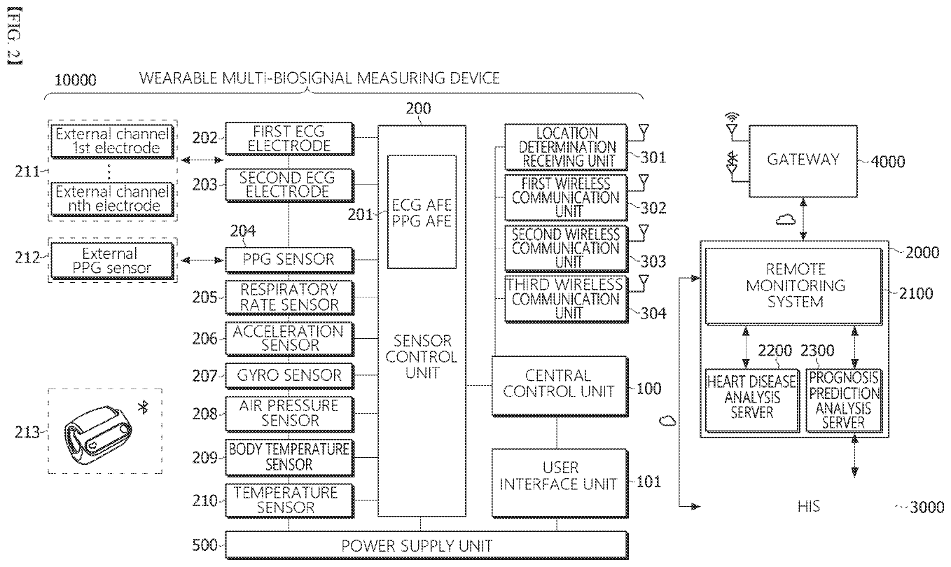

FIG. 2 is a block diagram showing a hardware configuration of a wearable multi-biometric measuring device 1000, a gateway, and a remote monitoring system according to an embodiment of the present invention.

Referring to FIG. 2, the wearable multi-biosignal measuring device 1000 includes a sensor control unit 200 connected to built-in sensor units 202 to 210, a central control unit 100, a user interface unit 101, a location determination receiving unit 301, first to third wireless communication units 302 to 304, and a power supply unit 500. External sensors 211 to 213 may be selectively detachably connected to the wearable multi-biosignal measuring device 1000.

The built-in sensor units 202 to 205 and 209 of the wearable multi-biosignal measuring device 1000 include one or more built-in sensors 202 to 210.

The built-in sensor includes a built-in ECG sensor. The built-in ECG sensor may measure an ECG of a patient's lead I through first and second ECG electrodes 202 and 203 exposed to the outside of the wearable multi-biosignal measuring device 1000. The ECG electrodes may be interpreted as ECG electrodes.

The first ECG electrode 202 is a left hand electrode LA that is in contact with the patient's left hand. The second ECG electrode 203 is a right hand electrode RA that comes into contact with the patient's right hand.

When a patient wears the wearable multi-biosignal measuring device 1000, the wrist of the patient comes into contact with the first ECG electrode 202. The first ECG electrode 202 has two electrode surfaces, which are the same electrode surfaces connected in a circuit, so that more reliable contact is made on the wrist during measurement. When the patient touches the second ECG electrode 203 with the finger of the opposite hand while wearing the wearable multi-biosignal measuring device 1000, an ECG signal of lead I may be obtained through the built-in ECG electrodes 202 and 203.

The built-in sensors 204 to 210 of the wearable multi-biosignal measuring device 1000 may include a photoplethysmography (PPG) sensor 204, a respiratory rate sensor 205, an acceleration sensor (an accelerator) 206, and a gyro sensor (a gyrometer) 207, an air pressure sensor (a barometer) 208, a body temperature sensor 209, and a temperature sensor 210. The built-in sensors are not limited to FIG. 1, and some sensors may be omitted or other sensors may be added.

The PPG sensor 204 measures whether it is worn, a pulse wave signal, and an oxygen saturation using a light emitter (a light emitting diode, LED) and a light receiver that face the wrist of the patient when the patient wears the wearable multi-biosignal measuring device on the wrist. In the case of blood pressure, it may be measured using a PPG signal output from the PPG sensor 204 and an ECG signal measured from the ECG electrodes 202 and 203.

The respiratory rate sensor 205 measures the respiratory rate through a change in body impedance of the patient when the patient wears the wearable multi-biosignal measuring device 1000 on the wrist.

The body temperature sensor 209 measures the skin temperature of the patient with the wearable multi-biosignal measuring device by emitting infrared rays to the patient's forehead.

The acceleration sensor 206 and the gyro sensor 207 recognize an exercise amount, a change in posture, and a sleep state of the patient wearing the wearable multi-biosignal measuring device 1000. For example, the remote monitoring system 2000 may detect a state of the patient whether the patient is walking, exercising, sleeping, or stationary based on output signals of the acceleration sensor 206 and the gyro sensor 207, and generate a notification message notifying an emergency situation, such as a fall.

The air pressure sensor 208 measures the air pressure level in the vicinity of the patient wearing the wearable multi-biosignal measuring device 1000. The temperature sensor 210 measures the temperature of the surrounding environment of the patient.

The sensor control unit 200 converts signals received from the ECG electrodes 202 and 203 and output signals of the sensors 204 to 210 into sensor values of digital signals, which are to be processed by the central control unit 100, and transmits the converted signals to the central control unit 100. The sensor control unit 200 includes an analog front end (AFE) 201.

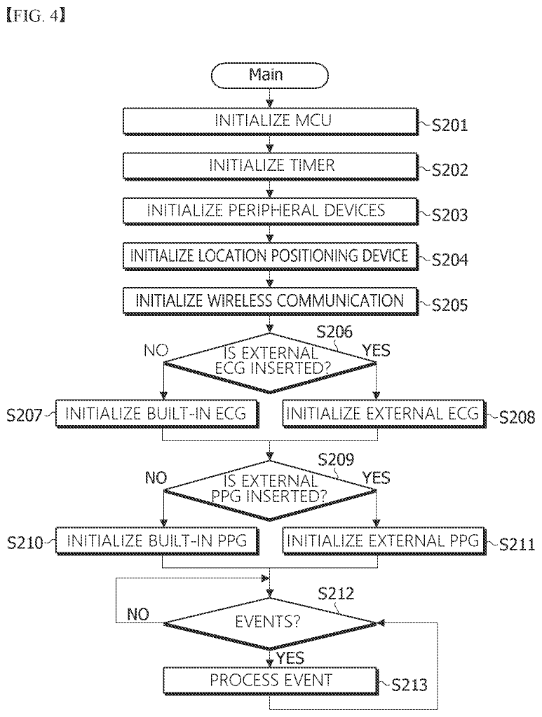

The AFE 201 includes a built-in ECG measurement unit (hereinafter referred to as a “built-in ECG AFE”) and a built-in oxygen saturation measurement unit (hereinafter referred to as a “built-in PPG AFE”) that are initialized and driven when external electrodes/sensors shown in FIG. 4 and FIGS. 4A and 4B are not connected to the wearable multi-biosignal measuring device 1000, and an external ECG measurement unit (hereinafter referred to as an “external ECG AFE”) and a built-in ECG measurement unit (hereinafter referred to as a “built-in PPG AFE”) that are initialized and driven when the external sensors are connected to the wearable multi-biosignal measuring device 1000.

The built-in ECG AFE amplifies an analog signal (an ECG measurement signal) received from the built-in ECG electrodes 202 and 203, removes noise from the signal, and converts the signal into a digital signal through an analog/digital (A/D) converter. The external ECG AFE amplifies an analog signal (an ECG measurement signal) received from the external ECG electrodes 221 to 224, removes noise from the signal, and converts the signal into a digital signal.

The built-in PPG AFE amplifies an output signal (a blood flow measurement signal of blood flowing through a blood vessel) of the light receiver, which is generated when light generated from the LED of the built-in PPG sensor 204 is emitted to the patient's wrist, removes noise from the signal, and then converts the signal into a digital signal. The external PPG AFE amplifies an output signal (a blood flow measurement signal of blood flowing through a blood vessel) of the light receiver, which is generated when the light generated from the LED of the external PPG sensor 225 is emitted to the patient's fingertip, removes the noise from the signal, and then converts the signal into a digital signal.

The digital signal output from the sensor control unit 200 is multi-biosignal measurement data including ECG measurement data, oxygen saturation measurement data, and measurement data for four vital signs (blood pressure, pulse rate, respiratory rate, and body temperature).

The central control unit 100 may include a micro control unit (MCU) for controlling signal processing and input/output, a timer, and a memory.

The central control unit 100 controls all components of the wearable multi-biosignal measuring device 1000. The central control unit 100 processes user input data received through the user interface unit 101. The central control unit 100 transmits the multi-biosignal measurement data received from the sensor control unit 200 to the remote monitoring system 2000 via a gateway through wireless communication units 302 to 304 as shown in FIG. 2. The central control unit 100 may provide data received from the remote monitoring system 2000, for example, message data, such as a control command or notification indicating a biosignal measurement cycle, a diagnosis result derived using an AI-based diagnosis algorithm, and the like to a data output part of the user interface unit 101 such that the data is displayed on the display. A control command instructing measurement of a biosignal may be generated from the remote monitoring system 2000.

The user interface unit 101 is connected to the central control unit 100. The user interface unit 101 includes a user data input part and a data output part. The user data input part receives a user input through the user data input part, for example, a button or a touch screen. The data output part may display an operating state of the wearable multi-biosignal measuring device 1000 and a biosignal measurement value of a patient in real time, and may output an alarm signal or a message as a sound signal. The biosignal measurement value may be displayed in the form of a preset graph. The data output part may include a display implemented as a liquid crystal display (LCD) or an organic light-emitting diode (OLED) display. In addition, the data output part may include a speaker outputting a sound signal, a vibration generator, and the like.

The location determination receiving unit 301 receives location signals from the multi-biosignal measurer. The central control unit 100 transmits location information of the multi-biosignal measuring device 1000 from the location determination receiving unit 301 to the remote monitoring system 2000 through the wireless communication units 302 to 304. The location determination receiving unit 301 receives location information through a global positioning system (GPS) in the case of outdoors and receives indoor-based location information in the case of indoors in which GPS reception is not possible. The remote monitoring system 2000 may receive a coordinate signal and monitor the location of a patient in a hospital or the location of a discharged patient in real time. In the case of using a smart phone as a gateway, location information may be obtained by utilizing the location information of the smart phone, and the above process may be omitted.

The wireless communication units 302 to 304 are connected to the central control unit 100 and transmit biosignal measurement signals received from the central control unit 100 to the remote monitoring system 2000 through the gateway 4000. The first wireless communication unit 302 is a communication unit for accessing the remote monitoring system 2000 without going through a user's smart phone, and may include for example, a wireless-fidelity (WiFi) network, a narrow band Internet of Things (NB-IoT) network, or a Long Term Evolution (LTE) network. The second wireless communication unit 303 is a communication unit for when a user's smart phone is used as a gateway, and in this case access is made through Bluetooth Low Energy (BLE) communication. The third wireless communication unit 304 may, in order to register or cancel the multi-biosignal measuring device 1000 in the remote monitoring system 2000 when hospitalized or discharged, use NFC or RFID communication. In this case, patient information and measurement cycle setting data of the remote monitoring system 2000 are transmitted to the multi-biosignal measuring device 1000, and a unique identification (ID) code of the multi-biosignal measuring device 1000 is transmitted to the remote monitoring system 2000.

The power supply unit 500 is connected to the built-in sensor units 221 to 210, the sensor control unit 200, the central control unit 100, the user interface unit 101, the location determination receiving unit 301, and the wireless communication units 302 to 304, and supplies power required to drive such driving elements. The power supply unit 500 may include a battery, a battery charging circuit, a DC adapter, and a Universal Serial Bus (USB) port.

When external sensors 221 to 226 are connected to the multi-biosignal measuring device 1000, the sensor control unit 200 detects a change in potential of ports, to which the external sensors 221 to 226 are connected, and generates an interrupt signal to determine whether connection of the external sensors 221 to 226 is made. One or more external ports to which the external sensors 221 to 226 are connected are exposed in the form of a jack or a USB connector on the side of the multi-biosignal measuring device 1000, and a plurality of external sensors share the same port.

In addition, the external ECG electrode 211 may include a unique identification (ID) code according to a measurable lead. The external ECG electrode 211 may include n (n is a natural number greater than or equal to 2) external channel electrodes. The central control unit 100 may distinguish the type of external ECG electrode 211 according to the unique ID code and recognize different external ECG channels.

The central control unit 100, in response to the external ECG electrode 211 or the external PPG sensor 212 being connected to the multi-biosignal measuring device 1000, disables the built-in sensor corresponding thereto, and converts a signal received from the external sensor 211 or 212 into a digital signal.

The external sensors 211 to 213 are not provided for general patients with low severity of illness, but are used when more precise biometric measurement is required according to the lesion or prognosis of a patient.

Compared to built-in ECG electrodes, which are capable of only measuring ECG signals of lead I, external ECG electrodes are capable of selectively enabling not only limb leads (leads I, II, and III or aVr, aVl, and aVf) but also chest leads v1 to v6 depending on the type of external ECG electrode, and thus more extensive ECG data may be obtained, and a wide range of heart diseases may be distinguished.

Compared to built-in PPG sensors that perform measurement on the wrist, in which there is a difficulty in blocking ambient light, and a lot of noise components causing errors in the measurement results are generated, external PPG sensors use a finger clip and thus, ambient light may be effectively blocked and high quality pulse wave signals may be obtained.

The display of the user interface unit 101 may employ an organic light-emitting diode (OLED) display. The user interface unit 101 may further include a touch screen, an internal speaker, and a vibrator for user input.

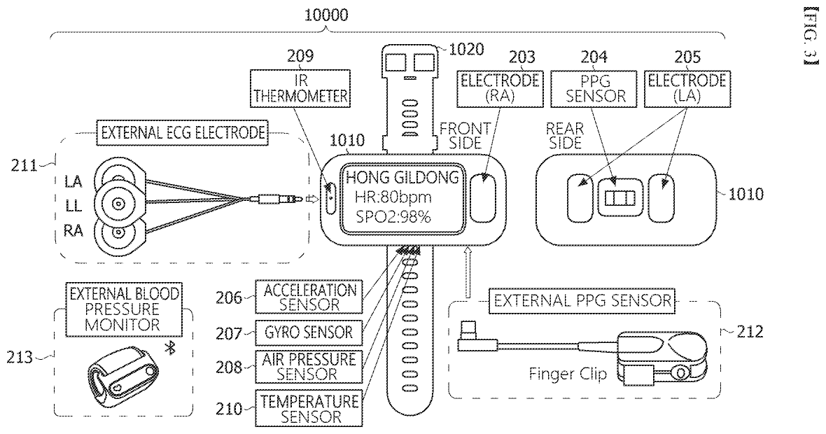

FIG. 3 is a diagram showing a multi-biosignal measuring device 1000 in a wearable form according to an embodiment of the present invention.

Referring to FIG. 3, the multi-biosignal measuring device 1000 may be implemented in a main body 1010. A band 1020 may be connected to the main body 1010 and wound around a user's wrist.

The main body 1010 includes a device lower part (or a rear side) which faces the user's wrist when the wristband is wound around the user's wrist and at which the built-in oxygen saturation sensor 204 and the left hand electrode (or a first electrode, 202) of the built-in ECG sensor are exposed, and a device upper part (or a front side) at which the built-in body temperature sensor 209, the right hand electrode (or a second electrode, 203) of the built-in ECG sensor, and the display of the user interface unit 101 are exposed.

The built-in sensors may include the built-in body temperature sensor 209 applicable as an infrared (IR) thermometer, the acceleration sensor 206, the gyro sensor 207, the air pressure sensor 208, the ambient temperature sensor 210, and the like.

The built-in ECG electrodes 202 to 203 are disposed with the right hand (right ankle; RA) electrode 203 on the upper part (the front side) of the device and the left hand (left ankle; LA) electrode 202 on the lower part (the rear side) of the device such that a lead I signal is measurable. The left hand (left ankle) electrode 202 has two electrode surfaces separated on both sides of the built-in PPG sensor, which are the same electrode surfaces connected in a circuit, so that stable measurement is possible when measuring an ECG.

In addition, bio-impedance may also be measured from the ECG electrodes 202 to 203, so that the respiratory rate may be measured through the bio-impedance that changes during inhalation and exhalation.

The built-in PPG sensor 204 is disposed on the lower part (the rear side) of the device in a structure that is recessed into the device to prevent penetration of ambient light as much as possible, and it is possible to employ a sensor including an IR LED for determining a wearing state to determine whether the wearable multi-biosignal measuring device 1000 is being worn.

The external ECG electrode 211 is an example using a 5-pole ear-jack type connector, which is used for a right ankle (RA) signal, a left ankle (LA) signal, a left leg (LL) signal, an attaching/detaching signal, and a unique ID code. Accordingly, three types of limb lead signals, from lead I to lead III may be read.

The external PPG sensor 212 is an example in a form of a finger clip structure that allows ambient light to be effectively blocked. In this case, a USB C-Type connector is used, which is an example designed to share a connector with a charger.

The external blood pressure monitor 213 is illustrated as a wireless cuff type blood pressure monitor, in which the multi-biosignal measuring device is capable of receiving a measurement result through BLE communication.

FIG. 4 is a flowchart showing a power-on sequence immediately after power is applied to the wearable multi-biosignal measuring device 1000 shown in FIG. 3.

When a user selects a power ON button in the user interface unit 101, power is supplied to the central control unit 100 from the power supply unit 500, and a MCU starts operating. First, the MCU initializes various internal registers of the MCU itself or internal devices of the MCU (S201), and generates and initializes various timers required for system operation (S202). In addition, the MCU initializes various peripheral devices in a control board (S203) and initializes devices for positioning (S204), and initializes wireless communication functions, such as BLE, WiFi, and NFC/RFID (S205). Thereafter, the MCU determines whether an external extension sensor currently attached to the system is inserted (S207 and 209) to perform initialization on built-in or external sensors (S207, S208, S210, and S211), and enters an infinite loop. In the infinite loop, the MCU operates in a structure that continuously checks an event which is to be currently processed (S212) and when an event occurs, calls a corresponding event handler (S213) and processes the event.

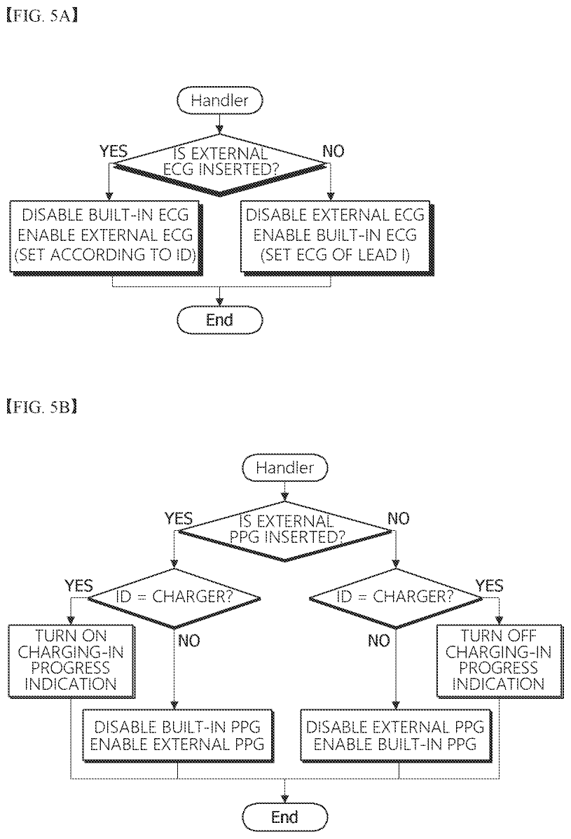

FIGS. 5A and 5B are flowcharts showing interrupt service routines depending on whether external sensors are connected.

In response to an external ECG electrode being inserted, a general purpose input output (GPIO) port for detecting an external ECG electrode changes from level “H” to “L,” and the sensor control unit 200 detects this change and generates an interrupt. In this case, an interrupt handler shown in FIG. 5A is called and executed. The interrupt handler checks an insertion state of an external ECG electrode, and since it is in an inserted state, disables the current built-in ECG and enables the external ECG. In addition, the interrupt handler reads a unique ID code of the inserted external ECG electrode, and allocates ECG channels of RA, LA, and LL. Even in response to the inserted external ECG being removed, the GPIO port for detecting an external ECG sensor changes from level “L” to “H” and an interrupt is generated, and thus the interrupt handler shown in FIG. 5A is executed in the same manner, and since the external ECG electrode is a removed state, the current external ECG is disabled and the built-in ECG is enabled. In this case, ECG channels are allocated for two channels of RA and LA.

Interface pins of the charger and the external sensors are each provided with an ID code for distinguishing an inserted external device. The ID code is a value for distinguishing each of the external sensors, and is preset in each of the charger and the external sensors to distinguish the external sensor. When a charger is connected to an external PPG port, the ID code of the charger is read through pins of the external PPG port. The sensor control unit 200 detects a voltage change of the pins of the external PPG port and generates an interrupt, and the central control unit compares an ID received through the pins of the external PPG port with a preset ID to determine whether a charger is connected, and the type of connected external sensor.

In response to a charger or an external PPG sensor 212 being inserted into an external PPG port, a GPIO port for detecting the external PPG sensor 212 changes from level “H” to “L” and thus an interrupt is generated from the sensor control unit 200.

In this case, an interrupt handler shown in FIG. 5B is called and executed. The interrupt handler checks an insertion state of the external PPG sensor 212, and since it is in an inserted state, reads an unique ID code read, and in the case of level “H,” performs an charging in progress indication, and the handler ends. However, when the unique ID code is “L,” which indicates that it is an inserted state of the external PPG, the current built-in PPG sensor 204 is disabled, and the external PPG sensor 212 is enabled. In response to removal of the inserted external PPG sensor 212 of the charger, the GPIO port for detecting the external PPG sensor 212 changes from level “L” to “H,” and an interrupt is generated, and thus the interrupt handler shown in FIG. 5B is executed. Since it is a removed state of the external PPG sensor 212 or the charger, the external PPG sensor 212, if enabled, is disabled, and the built-in PPG sensor 204 is enabled, and the charging in progress indication is turned off.

The external ECG electrodes, which have varying lead signals, have a unique ID code as well as a signal informing attachment and detachment of an external electrode, and thus the ID code identifies from which lead, among limb leads Lead I-III, aVr, aVl, and aVf, and chest leads v1 to v6, each channel signal originates. Table 1 below is an example of ID codes for external ECG electrodes. It should be noted that the ID codes are not limited to Table 1.

TABLE

| ID | Number of channels | ECG channel | |
|---|---|---|---|
| ‘000’ | One channel(Lead I) | CH1 | Lead I-N electrode |
| | | CH2 | Lead I-P electrode |
| ‘101’ | Three channels (Lead I~Lead III) | CH1 | Lead I-N electrode |
| | | CH2 | Lead I-P electrode |
| | | CH3 | LL(Left Leg) |
| ‘110’ | Five channels (Lead I~Lead III, | CH1 | RA(Right Ankle) |
| | v1, v6) | CH2 | LA(Left Ankle) |
| | | CH3 | LL(Left Leg) |
| | | CH4 | V1 |
| | | CH5 | V6 |

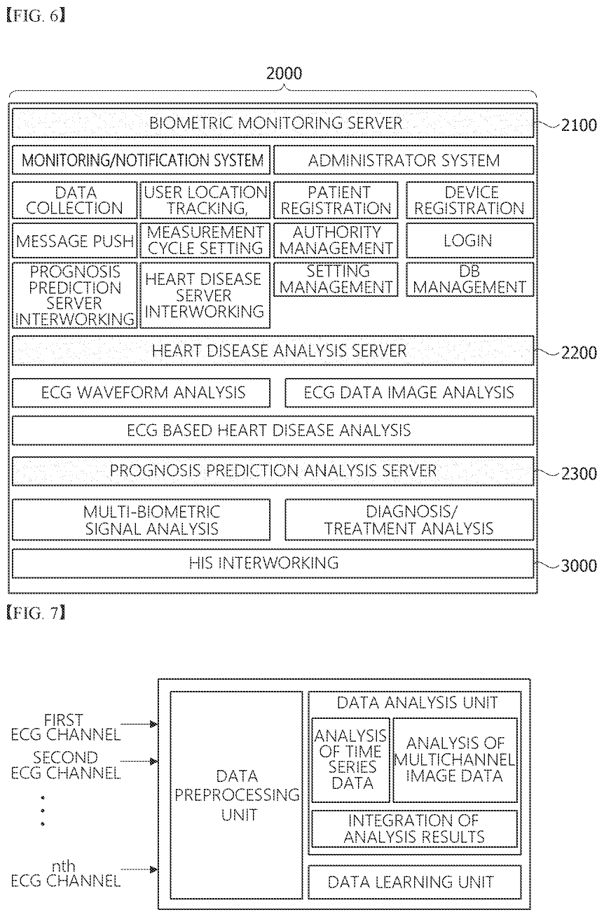

FIG. 6 is a diagram showing the configuration of a remote monitoring system 2000 in detail.

Referring to FIG. 6, the remote monitoring system 2000 includes a biometric monitoring server 2100, a heart disease analysis server 2200, and a prognosis prediction analysis server 2300.

The biometric monitoring server 2100 performs functions of rendering a trend change of biometric data transmitted through the gateway in real time, receiving a prognosis prediction analysis result, which is obtained by analysis on the biometric data in association with a heart disease analysis result received through the heart disease analysis server 2200 and the HIS, from the prognosis prediction analysis server 2300 to generate or transmitting the prognosis prediction analysis result.

The biometric monitoring server 2100 performs a function of displaying changes in biometric information of a registered patient so that the medical staff can monitor a biometric data trend change as well as a function of registering a patient requiring monitoring, or registering a device for the patient, and performs a function of log-in management or transmission or reception of various set values to or from the multi-biosignal measuring device 1000. The biometric monitoring server 2100 may include a monitoring/notification system and an administrator system. The monitoring/notification system may serve to perform data collection, user location tracking, a message push service, measurement cycle setting, prognosis prediction server interworking, and heart disease server interworking. The administrator system may serve to perform patient registration, device registration, authority management, login, setting management, and DB management.

The heart disease analysis server 2200 performs a heart disease analysis function by running a lead I waveform analysis algorithm based on ECG signals, which are received from the multi-biosignal measuring device 1000 through the gateway 4000, through the biometric monitoring server 2100 to analyze an ECG data waveform, and by running a redundant algorithm of AI ECG image analysis to analyze a heart disease. The multi-biosignal measuring device 1000 with the external ECG electrode 211 inserted thereinto performs a multiple channel heart disease analysis function to correspond to the channel of the external ECG electrode.

The prognosis prediction analysis server 2300 interworks with the HIS 3000 to check a medical history or treatment history of a patient, and performs a prognosis prediction analysis function and a risk prediction function for a disease based on heart disease information from the heart disease analysis server 2200 and real-time biometric information from the biometric monitoring server 20001.

The heart disease analysis server 2200 and the prognosis prediction analysis server 2300 may be separated from the remote monitoring system 2000 and present in a cloud.

FIG. 7 is a diagram showing an artificial intelligence-based diagnosis algorithm for built-in or external ECG sensor data. The heart disease analysis server 2200 may predict a heart disease by driving an AI-based diagnosis algorithm. The heart disease analysis server 2200 may be included in or separated from the remote monitoring system 2000.

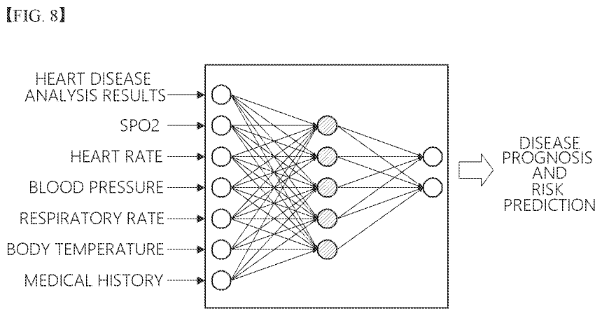

FIG. 8 is a block diagram showing a configuration of a prognosis prediction server that informs a prognosis prediction result for diseases by learning multiple biosignals, a heart disease analysis result, and a medical history. The prognosis prediction server may be included in or separately installed from the remote monitoring system 2000.

Multi-Biosignal Measurement Method Using General Patient Device

In the case of a device for general patients, when a patient wears the multi-biosignal measuring device 1000 on the wrist, multiple biosignals may be automatically measured in an unaware state in a non-invasive manner according to a measurement cycle provided by the biometric monitoring server 2100. A respiratory rate, a single-channel (or Lead I) ECG, and a body temperature may be directly measured by a user (the patient) when a notification sound is output from the wearable multi-biosignal measuring device 1000 or a notification message is received from the wearable multi-biosignal measuring device 1000 according to the measurement cycle.

In order to increase test accuracy by keeping the posture of the patient stable during non-awareness measurement, a notification message of a preset measurement time is displayed on the display of the multi-biosignal measuring device 1000 together with a notification sound, and when the stable posture of the patient is kept for a predetermined time or longer, a non-awareness measurement is automatically performed. The posture of the patient may be detected in real time by the acceleration sensor 206 and the gyro sensor 207.

Upon receiving, by the multi-biosignal measuring device 1000, a notification message requesting measurement of a body temperature, placing an external thermometer, e.g., a non-contact thermometer, for measuring the temperature of the forehead, on the forehead of the patient allows the temperature to be automatically measured using an IR sensor and recorded in the server. Upon receiving an ECG or respiration rate measurement notification message, placing a finger of a hand opposite to a hand, on which the multi-biosignal measuring device 1000 is worn, on a second ECG electrode 203 for 30 seconds allows a single-channel ECG and a respiration rate to be automatically measured and recorded in the server.

Information about an exercise amount and sleep of the patient are checked in real time using the acceleration sensor 206 and the gyro sensor 207, and daily activity statistics and sleep information are provided through a dedicated application (APP) pre-installed on the mobile terminal of the patient such that the daily activity statistics and sleep information along with the measurement results of multiple biosignals are provided in real time to the mobile terminals of the patient and the medical staff and the monitoring system of the hospital.

Multi-Biosignal Measurement Method Using Device for Patients with Moderately-Severe or Severe Symptoms

In the case of a device for patients with moderately-severe or severe symptoms, a 3-lead ECG patch to which external ECG electrodes and/or an external PPG sensor are connected may be provided according to the severity of symptoms of the patient along with the general patient device.

In the device for patients with moderately-severe or severe symptoms, basic multi-biosignals are measured at preset intervals in the same manner as in the device for general patients. Additionally, upon receiving, by the multi-biosignal measuring device 1000, an ECG measurement notification message according to a biosignal measurement set time, attaching the ECG patch connected to the external ECG electrodes to the chest allows 3-lead ECG measurement data to be automatically recorded in the server. In addition, upon receiving, by the external ECG electrode multi-biosignal measuring device 1000, an oxygen saturation measurement message, attaching the external PPG sensor to the finger allows oxygen saturation measurement data measured by the external PPG sensor to be automatically recorded in the server.

The embodiments of the present invention have been described in more detail with reference to the accompanying drawings, but the present invention is not necessarily limited to these embodiments, and may be variously modified without departing from the technical spirit of the present invention. Therefore, the embodiments disclosed in the present invention are not intended to limit the technical spirit of the present invention, but to explain, and the scope of the technical spirit of the present invention is not limited by these embodiments. Therefore, it should be understood that the embodiments described above are illustrative in all respects and not restrictive. The scope of protection of the present invention should be interpreted by the claims below, and all technical spirits within the equivalent range should be interpreted as being included in the scope of the present invention.

### INDUSTRIAL APPLICABILITY

According to the present invention, multiple biosignals can be measured in an unaware state using a wearable multi-biosignal measuring device individually provided to a severely ill hospitalized patient or a discharged patient who needs prognosis management, and the patient can measure required biosignals him/herself. In addition, the remote monitoring system is connected to the wearable multi-biosignal measuring device through a wireless communication network, and thus continuously monitors multiple biosignals received from the wearable multi-biosignal measuring device in real time. Accordingly, in the case of a severely ill hospitalized patient, measurement and monitoring of multiple biosignals can be performed in a non-contact manner, and in the case of a discharged patient who requires prognosis management, prognosis management can be remotely performed without visiting the hospital.

1. A wearable device for measuring multiple biosignals, comprising:
a main body having one or more external ports;
a wristband connected to the main body and wound around a wrist of a user; and
one or more external sensors connected to the external ports,
wherein the main body includes:
one or more built-in sensors configured to measure biosignals;
a wireless communication unit configured to transmit multiple biosignals obtained from the built-in sensors or the external sensors to an outside;
a user interface unit configured to display a notification message instructing measurement of the multiple biosignals and receive user data;
a sensor control unit configured to amplify an output signal of the built-in sensor and convert the amplified signal into a digital signal, detect whether the external sensor is connected to the external port, and when the external sensor is connected to the external port, disable the built-in sensor that measures the same biosignal as the external port; and
a central control unit configured to supply multi-biosignal data from the sensor control unit to the wireless communication unit and transmit a notification message received from the wireless communication unit to a display of the user interface unit.

2. The wearable device of claim 1, wherein:
the built-in sensors include a built-in electrocardiogram (ECG) sensor, a built-in oxygen saturation sensor, a built-in respiratory rate sensor, a built-in accelerometer sensor, a built-in gyro sensor, a built-in air pressure sensor, a built-in body temperature sensor, and a built-in temperature sensor; and
the external sensor includes one or more of an external electrocardiogram patch, to which external ECG electrodes are connected, an external oxygen saturation sensor, an external blood pressure monitor, and an external body temperature sensor.

3. The wearable device of claim 2, wherein the main body includes:
a rear surface that faces the wrist of the user while the wristband is wound around the wrist of the user and through which the built-in oxygen saturation sensor and a first electrode of the built-in ECG sensor are exposed; and
a front surface at which the built-in body temperature sensor, a second electrode of the built-in ECG sensor, and the display of the user interface unit are exposed.

4. The wearable device of claim 2, wherein the sensor control unit is configured to:
when the external ECG electrodes are connected to the external port, disable a built-in ECG measuring unit connected to the built-in ECG electrodes and enable an external ECG measuring unit to which the external ECG electrodes are connected; and
when the PPG sensor is connected to the external port, disable a built-in PPG measuring unit connected to the built-in PPG sensor and enable a built-in PPG measuring unit to which the external PPG sensor is connected.

5. A remote monitoring system connected to a multi-bio signal measuring device through a wireless communication network, the multi-biosignal measuring device including a main body having one or more external ports, a wristband connected to the main body and wound around a wrist of a user, and one or more external sensors connected to the external ports, wherein the remote monitoring system is configured to:
display, in a biometric monitoring server, multiple biosignals received from the multi-biosignal measuring device in time series using a graph;
transmit a received electrocardiogram (ECG) signal to an artificial intelligence (AI)-based heart disease analysis server and receive an analysis result for a heart disease;
receive a prognosis prediction analysis result obtained through AI learning based on the received multiple biosignals, the analysis result of the heart disease analysis server, and a medical history of a hospital information system; and
analyze data received from the multi-biosignal measuring device to monitor an activity level, sleep, sleep apnea, and a fall of a user wearing the multi-biosignal measuring device in real time, and determine a location of the user and whether the user departs from a quarantine area.

6. The remote monitoring system of claim 5, wherein the remote monitoring system, in response to the multi-biosignal measuring device being in contact with a near field communication (NFC)/radio frequency identification (RFID) reader, transmits a profile message including patient information, a type of biosignal, a measurement cycle of a biosignal, and a quarantine range to the multi-biosignal measuring device.

7. The remote monitoring system of claim 5, wherein the remote monitoring system issues an alarm in response to a result of a prognosis prediction exceeding a reference value set for each patient, transmits an alarm message of the result to a mobile terminal of a medical staff or a multi-biosignal measuring device of a patient, and allows a message to be transmitted or received between the medical staff and the patient.

8. The remote monitoring system of claim 5, wherein the remote monitoring system transmits, to the multi-biosignal measuring device, a positioning request signal for a location to monitor whether a patient has departed from a predetermined location range.

9. The remote monitoring system of claim 5, wherein a type or a measurement cycle of a biosignal of the multi-biosignal measuring device is remotely set according to a severity of symptoms or prognosis of a patient in a manual or automatic manner.

10. The wearable device of claim 3, wherein the sensor control unit is configured to:
when the external ECG electrodes are connected to the external port, disable a built-in ECG measuring unit connected to the built-in ECG electrodes and enable an external ECG measuring unit to which the external ECG electrodes are connected; and
when the PPG sensor is connected to the external port, disable a built-in PPG measuring unit connected to the built-in PPG sensor and enable a built-in PPG measuring unit to which the external PPG sensor is connected.
